(19) United States
(12) Patent Application Publication
(10) Pub. No.: US 2020/0260966 A1
(43) Pub. Date: Aug. 20, 2020

(54) MODULAR WEARABLE SENSOR

(71) Applicant: BraveHeart Wireless Inc., Nashua, NH (US)

(72) Inventors: Stephen A McCalmont, Hollis, NH (US); Stuart P. MacEachern, Hopkinton, MA (US)

(21) Appl. No.: 16/591,882

(22) Filed: Oct. 3, 2019

Related U.S. Application Data
(60) Provisional application No. 62/807,348, filed on Feb. 19, 2019.

(51) Int. Cl. A61B 5/0205; A61B 5/1455; A61B 5/0408; A61B 5/00; A61B 5/01; A61B 5/11; A61B 5/145

(52) U.S. Cl. CPC A61B 5/02055; A61B 5/14552; A61B 5/0408; A61B 5/6833; A61B 5/01; A61B 5/0533; A61B 5/1116; A61B 5/14532; A61B 5/7475; A61B 5/746; A61B 5/11

(57) ABSTRACT

A modular wearable health sensor having a substantially flexible attachment means connected to a housing comprising a power source and at least one processor through a relatively rigid and narrow spine disposed substantially centrally thereon having a variety of clinical and non-clinical uses that provides more comfortable and durable attachment of biometric sensors to a user by allowing the attachment means to contour to the user's body without the housing also being forced to do so along its entire width.

### RELATED APPLICATIONS

This application claims the benefit of U.S. Provisional Application No. 62/807,348, filed Feb. 19, 2019. This application is herein incorporated by reference in its entirety for all purposes.

### FIELD OF THE INVENTION

The invention relates to medical sensors and, more particularly, to modular, fully-featured wearable medical sensors suitable for use in clinical and non-clinical environments.

### BACKGROUND OF THE INVENTION

The wearable medical device market has expanded greatly over the past decade, with consumer devices, such as the Fitbit® and Jawbone® wireless activity trackers, becoming a popular way for people to quantify and take charge of their personal fitness and overall well-being. While these devices are the most visible to consumers, rapid developments are simultaneously occurring in similar devices having a wide range of clinical uses. These devices are constantly becoming smaller, offering better battery life through both new battery chemistries and more efficient electronics, while providing more data and using better and more efficient algorithms to render that data useful. Existing clinical and other full-featured devices, however, are still relatively bulky and, as a consequence, uncomfortable to wear.

Although some clinical devices can be worn as a wristwatch might be worn, dependent on the biometric function(s) desired to be monitored, positioning of a wearable sensor, or even multiple wearable sensors, in various, specific locations on the body may be required. This is currently accomplished through the use of temporary adhesives, similar to those used in bandages. The form factor of such devices is generally similar to a medium-sized bandage.

The sensors and associated circuitry of such devices are typically either secured to the bandage-like adhesive or contained within it. As the circuit boards and other electronic components of prior art devices are at least semi-rigid, this results in the device, during use, exerting continuous outward (i.e. away from the user) pressure on the adhesive when affixed to a curved portion of the user's body. This results in an uncomfortable pulling sensation on the user while also causing the sensor to tend to detach therefrom. This tendency is exacerbated by the user's movements, which result in spikes of outward pressure due to movement-related minor changes in the curvature of the portion of the user's body onto which the device is affixed.

As the adhesive, being a temporary adhesive, weakens over time, these forces ultimately cause the adhesive to fail, often prior to the time at which the sensor was intended to be removed. Such a failure results in data loss in addition to a reduction in the cost/benefit ratio of the devices generally. In many cases, this failure also requires a new sensor to be placed on the user, which may require the user to visit a medical facility, at the very least resulting in a significant inconvenience. In some cases, such a failure may even trigger an alarm, potentially diverting medical resources from a true emergency.

The use of stronger adhesives would potentially resolve issues relating to the adhesive failing early, but would likely also result in additional discomfort to the user upon sensor removal and would not relieve the general discomfort associated with the use of such sensors.

The use of flexible circuit boards would also tend to ameliorate some of these issues, but would also increase the cost of such devices and potentially limit which sensors could be installed thereon.

What is needed, therefore, are techniques for making such devices more durable without decreasing their current capabilities while increasing user comfort.

### SUMMARY OF THE INVENTION

An objective of embodiments of the present disclosure is to provide a wearable health sensor that is modular, allowing for the majority of circuitry to be separated from the adhesive portion thereof.

A further objective of embodiments of the present disclosure is to reduce the amount of circuitry disposed on the adhesive portion of the wearable health sensor described herein, allowing the wearable health sensor to better contour to a user's body.

Still another object of embodiments of the present invention is to provide a wearable health sensor of improved durability and reusability.

Still even another object of embodiments of the present invention is to improve the ease of use of wearable health sensors, generally.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a top, elevation view of a wearable health sensor, configured in accordance with embodiments of the present disclosure;

FIG. 2A is a top, elevation view of the circuitry-containing portion of a wearable health sensor, in accordance with embodiments of the present disclosure;

FIG. 2B is a top, elevation view of the adhesive portion of a wearable health sensor, in accordance with embodiments of the present disclosure; and

FIG. 3 is side, elevation view of a wearable health sensor wherein the adhesive portion thereof is being flexed, in accordance with embodiments of the present disclosure.

### DETAILED DESCRIPTION

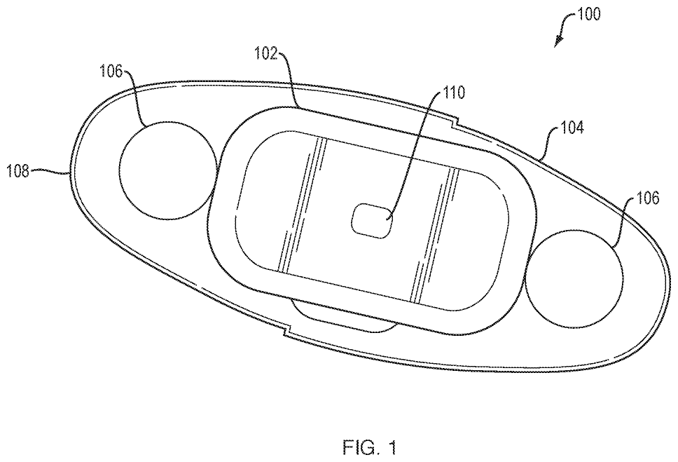

Now referring to FIG. 1, a top, elevation view of a wearable health sensor 100, configured in accordance with embodiments of the present disclosure, is shown. The wearable health sensor 100 includes a housing 102 containing circuitry necessary to the operation of the sensor. The wearable health sensor 100 further comprises a mounting strip 104, in embodiments similar in size and shape to a medium sized adhesive bandage, onto which the housing 102 can be affixed. The mounting strip 104 is used to attach the housing 102 to a user and, in embodiments, comprises an adhesive layer disposed opposite the housing 102 such that the mounting strip 104 may be removably attached to a user in any convenient location.

In embodiments, the mounting strip comprises electrodes 106 in operative communication with the housing 102, when affixed to the mounting strip 104, allowing circuitry contained therein to use the electrodes to monitor biometric data of a user therethrough.

In embodiments, the mounting strip 104 comprises a release liner 108 disposed on the adhesive portion(s) thereof, to ensure the adhesive is not contaminated prior to use.

In embodiments, the housing 102 is reusable and contains a power supply. The power supply, in embodiments, is a rechargeable battery that may be recharged using inductive charging technology, a charging port, or other charging technologies, as would be known to one of ordinary skill in the art. In other embodiments, an internal disposable battery is user-replaceable. In still other embodiments, a capacitor is used as a power source, enabling rapid charging.

In embodiments, the housing 102 comprises a function button 110, which can be programmed to perform a variety of functions, as necessary or desired.

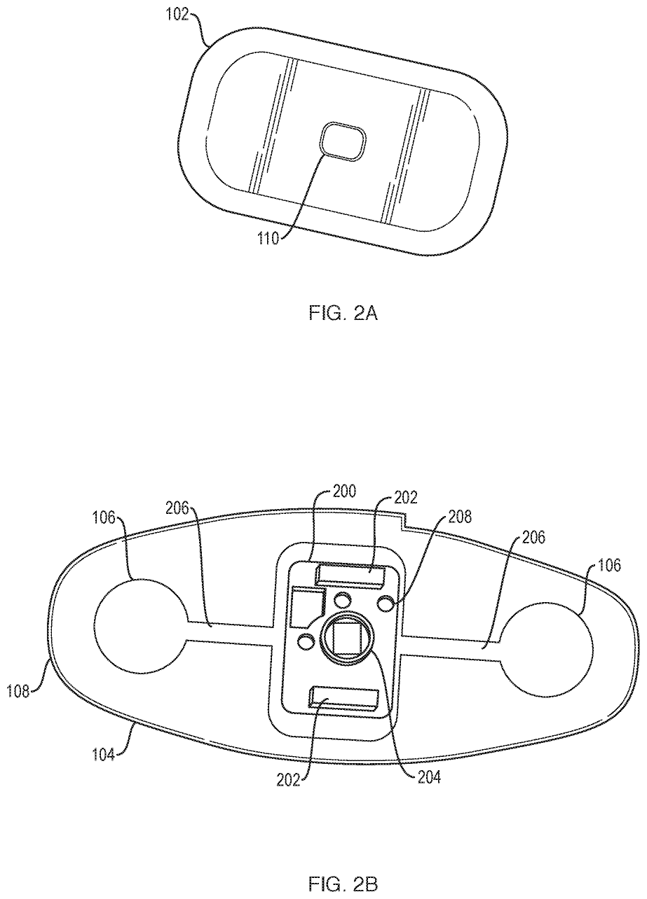

Now referring to FIG. 2A, a top, elevation view of the circuitry-containing portion of a wearable health sensor 100, in accordance with embodiments of the present disclosure, is shown.

FIG. 2B shows a top, elevation view of a mounting strip 104, in accordance with embodiments of the present disclosure, wherein the housing 102 has been removed therefrom. From this figure, it can be seen that the mounting strip comprises a relatively narrow spine 200 that is disposed substantially centrally on the mounting strip 104. Furthermore, the spine comprises a connector 204 disposed substantially centrally thereon. The connector 204 is configured to provide electrical connectivity between the housing 102 and mounting strip 104, which, in embodiments, contains a variety of sensors (e.g. electrodes 106) and/or pass-throughs for sensors contained within the housing 102.

For example, in embodiments, apertures 208 in the spine 200 of mounting strip 104 align with Light Emitting Diodes (LEDs) disposed on the bottom of the housing 102, allowing for the measurement of oxygen saturation in a user. In embodiments, thee apertures 208 are used to enable three frequency blood oxygen saturation measurement.

In embodiments, fiber optic wires, fiber optic cables, light pipes, and/or similar light-conveying means are disposed in the mounting strip 104 and positioned to align with light-emitting elements in the housing 102. Many additional sensor types could be used in conjunction with the wearable health sensor 100 described herein, as would be known to one of ordinary skill in the art.

In embodiments, the mounting strip 104 utilizes magnets 202 to secure the housing 102 thereto, utilizing corresponding magnetic materials disposed in the housing 102. In embodiments, these magnets 202 are phased magnets 202 that act to repel the housing 102 from the mounting strip 104 if the orientation of the two is incorrect (i.e. 180° off), discouraging users from assembling the components incorrectly.

The spine 200 is, in embodiments, connected to electrodes through flexible connections 206, which may be wires, traces, or other types of flexible connections, as would be known to one of ordinary skill in the art.

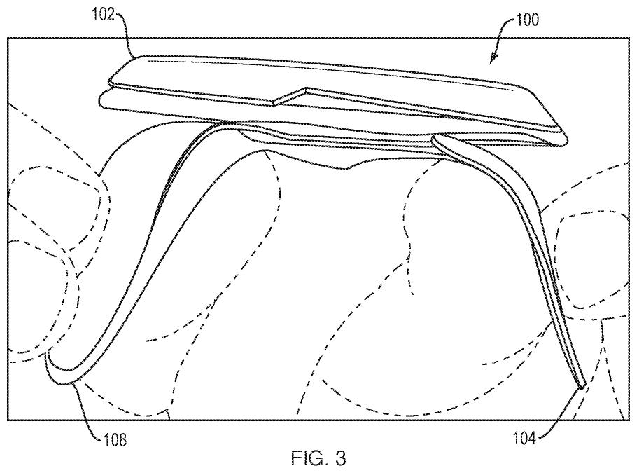

Now referring to FIG. 3, the flexibility of the present invention is demonstrated through an illustration of the mounting strip 104 and release liner 108 in a highly flexed position, wherein the housing 102 is shown not to substantially limit the flexibility of the mounting strip 104, due to its attachment thereto only using the relatively narrow spine 200 thereof. By limiting the width of the area of attachment of the housing 102 to the mounting strip 104, the flexibility of the mounting strip 104 is vastly improved, enhancing both user comfort and the ability of the mounting strip 104 to remain attached to a user for long periods of time, since the mounting strip 104 is allowed to more freely contour itself to the user. Furthermore, the housing 102 can be made larger than previously feasible, since it does not need to lie flat against a user's body (i.e. the mounting strip 104 is free to contour itself to a user's body independently of the housing 102).

The modularity of the present invention further reduces the waste involved with prior art health sensors, by allowing the replacement of mounting strips 104 that no longer retain the housing 102 to a user adequately while allowing the more expensive and environmentally harmful circuitry and power sources to be reused.

Embodiments further allow for a housing 102 to be mounted on a variety of mounting strips 104, each of which may contain different sensors and/or pass-throughs, thereby reducing their cost, compared to a mounting strip 104 that provides measurement capabilities that are not needed in a given situation.

Lastly, the modular wearable health sensor 100 described herein allows a user to easily remove the housing 102 when required, allowing them to engage in activities that may have been prevented when using prior art systems (e.g. a non-waterproof housing could be removed prior to showering or swimming, whereas prior art systems that are not removable from adhesive mounts might have to be covered or be made waterproof, resulting in additional expense).

The foregoing description of the embodiments of the invention has been presented for the purposes of illustration and description. Each and every page of this submission, and all contents thereon, however characterized, identified, or numbered, is considered a substantive part of this application for all purposes, irrespective of form or placement within the application. This specification is not intended to be exhaustive or to limit the invention to the precise form disclosed. Many modifications and variations are possible in light of this disclosure.

1. A modular wearable health monitor comprising:
a housing comprising a power source and at least one processor configured to process biometric data;
a flexible mount configured, during use, to be affixed to a user's body, the mount comprising a body attachment means for affixing said mount to the user's body; and
a substantially rigid spine disposed substantially centrally on said flexible mount, the spine comprising an attachment means for removably attaching said housing thereto,
wherein said spine is substantially narrower than the mount.

2. The modular wearable health monitor of claim 1 wherein said mount further comprises at least one sensor that, upon fixation of the housing to the spine, is put into electrical and/or optical communication with said housing.

3. The modular wearable health monitor of claim 1 wherein said spine further comprises apertures aligned with features of said housing.

4. The modular wearable health monitor of claim 3 wherein the features of said housing comprise Light Emitting Diodes.

5. The modular wearable health monitor of claim 4 wherein said light emitting diodes are in operative communication with at least one processor contained within said housing, wherein said processor is configured to utilize inputs therefrom to generate data corresponding to blood oxygen saturation measurements.

6. The modular wearable health monitor of claim 5 wherein said mount further comprises at least one EKG sensor configured to be in operative communication with said housing when attached thereto through said spine.

7. The modular wearable health monitor of claim 1 wherein said body attachment means comprises an adhesive.

8. The modular wearable health monitor of claim 7 wherein said adhesive is a pressure-sensitive adhesive.

9. The modular wearable health monitor of claim 7 wherein said adhesive is a waterproof adhesive.

10. The modular wearable health monitor of claim 1 wherein said modular wearable health monitor is configured to monitor heart rate, heart rate variability, steps taken, respiratory rate, blood oxygen levels, skin temperature, body posture, glucose levels, and galvanic skin response/electro dermal activity.

11. The modular wearable health monitor of claim 1 wherein said housing further comprises an inductive charging module configured to allow inductive charging of said power source.

12. The modular wearable health monitor of claim 1 wherein said housing further comprises a programmable button.

13. The modular wearable health monitor of claim 12 wherein said programmable button is configured, when activated, to perform a function selected from the group consisting of record data, alert emergency responders, and mark the data being recorded at that time for later review.
